(19) United States
(12) Patent Application Publication
(10) Pub. No.: US 2021/0313058 A1
(43) Pub. Date: Oct. 7, 2021

(54) MODULAR TELEHEALTH SYSTEM AND METHOD THEREOF

(71) Applicant: Zahid F. Mian, Loudonville, NY (US)

(72) Inventor: Zahid F. Mian, Loudonville, NY (US)

(21) Appl. No.: 17/219,996

(22) Filed: Apr. 1, 2021

Related U.S. Application Data
(60) Provisional application No. 63/003,406, filed on Apr. 1, 2020.

(51) Int. Cl. G16H 40/63; G16H 50/20

(52) U.S. Cl. CPC G16H 40/63; A61B 5/087; G16H 50/20

(57) ABSTRACT

A medical data acquisition device including multiple medical sensors located on a single device housing. The medical sensors can be operated to acquire multiple types of diagnostic data for a patient. The device can include a core component that can interface with each medical sensor and receive raw diagnostic data from each medical sensor. The core component can generate medical diagnostic data for the patient based on the raw diagnostic data provide the medical diagnostic data for the patient for processing by an assessment component. The device can be utilized in a system that includes a local computing device capable of performing an assessment using the medical diagnostic data. The device is configurable to have communications to medical evaluation methods such as a medical facility, medical system for artificial intelligence, or a medical expert system.

### REFERENCE TO RELATED APPLICATIONS

The current application claims the benefit of U.S. Provisional Application No. 63/003,406, filed on 1 Apr. 2020, which is hereby incorporated by reference herein.

### TECHNICAL FIELD

The disclosure relates generally to acquiring diagnostic data for a patient, and more particularly, to a device configured to acquire any of various types of medical diagnostic data.

### BACKGROUND OF THE INVENTION

In early 2020, the coronavirus-19 epidemic demonstrated the limitations of current medical resources when confronted with a pandemic. It is reasonable to assume, given the historical record, that this is not the last time such an event will occur.

The pandemic presented two major obstacles to doctors and patients. Firstly, the best method for preventing spread of disease is distancing—separating people from each other so that they cannot spread the disease. This in itself limited the practicality of having many people concentrate themselves at a doctor's office for diagnosis and treatment. Secondly, if a patient remained at home and tried to work with a doctor remotely, they had little-to-no access to the basic tools of a modern physician's diagnostic approaches—stethoscope, otoscope, blood pressure measurement (sphygmomanometer), SPO₂ sensors, and so on. This meant that in most cases all that a doctor could do is evaluate the patient based on verbal description of symptoms and, commonly, their temperature as provided by a home thermometer.

A similar set of challenges is confronted constantly by first responders in the field, and by military organizations worldwide, when faced with a victim of injury or illness and having no trained medical staff or medical equipment on hand.

Combined with existing videoconferencing, the existing technology does allow a trained physician to make some general diagnostic judgments, but falls far short of what would be expected from even a brief visit to an on-call urgent care center, let alone a fully-equipped hospital; at each of which the basic diagnostic tools and trained personnel would be readily available.

For example, standard sphygmomanometers for blood pressure detection are professional instruments that require specialized training. Home blood pressure measuring devices (such as those from Omron) exist, but are still fairly bulky, and are relatively rare. A large proportion of households will have at least one fever thermometer of some type, but only a very small proportion will have a blood pressure monitor.

Similarly, stethoscopes are specialty equipment used for heart and lung and sometimes gastrointestinal (GI) tract assessment. The average person cannot use them and won't have one in the house. Electronic stethoscopes exist, such as the CORE stethoscope from Eko, but are expensive and limited to sale to healthcare professionals.

An SPO₂ sensor is also a vital assessment tool (for blood oxygen content) but one rarely found in the home. However, these instruments are available in an inexpensive form and are likely found in more homes than sphygmomanometers and stethoscopes.

Examining the dilation/contraction of the eye, and other elements of the eye, is also important in diagnostics. There are some eye-focused home products, most notably the EyeQue, which performs an eye exam to determine the correction needed for glasses or contacts, but these are uncommon and not focused on medical applications, and are standalone.

Numerous tests, such as blood glucose levels, can be done in a doctor's office or urgent care center. Diabetics usually have a home glucose assessment device, but most people do not, and other testing is even less commonly available.

Other common diagnostic tools include things such as electrocardiograms (EKGs), radiographic imagery (X-ray, CT scan, etc.), pulmonology assessment such as spirometry, and others, few of which are even available for individuals and, if available, are rarely widely distributed.

Even if an individual had all of the needed equipment at home, they would not have any specific way to assess what the combination of readings might mean. Currently available systems for personal use contain little-to-no ability to perform analysis of the data. Additionally, current systems come in a variety of sizes and form factors that would make it infeasible for the average person to keep them handy, or even apply easily in a systematic fashion. Some prior art exists that attempts to address these issues, but all have shortcomings. For example, Neumann (U.S. Pat. No. 10,931,64361) describes a telehealth system which communicates with medical sensors at the patient, but assumes that communications are initiated by a medical professional remote from the patient, and does not describe the sensor module in a way that implies or requires that the module be able to support multiple compatible sensor devices, rather than being a single device of general description. The system also does not show or imply that the system local to the patient performs any analysis of the sensor data, nor that the system local to the patient can be used by the patient in the absence of a health professional's guidance.

Similarly, Demers and others in U.S. Pat. No. 10,478,261B2 describe a home telehealth kit that incorporates numerous sensors that may be linked to a central device such as a tablet, but focuses on the structural design of the kit, and neither requires nor implies that the sensor devices are or could be designed as integral parts of the system, using common electronics; nor does the kit's central device appear to perform any actual analysis of the sensor data, either singly (noting that the patient's temperature is elevated) or in combination (noting that the elevated temperature combined with a reddened throat with obvious white patches may indicate strep throat).

### SUMMARY OF THE INVENTION

Aspects of the invention provide a medical data acquisition device including multiple medical sensors located on a single device housing. The medical sensors can be operated to acquire multiple types of diagnostic data for a patient. The device can include a core component that can interface with each medical sensor and receive raw diagnostic data from each medical sensor. The core component can generate medical diagnostic data for the patient based on the raw diagnostic data provide the medical diagnostic data for the patient for processing by an assessment component. The device can be utilized in a system that includes a local computing device capable of performing an assessment using the medical diagnostic data.

Aspects of the invention described herein can overcome the limitations of current art designs for telemedicine (remote medical diagnostics and treatment) in the general population. The inventors recognize that current art often involves little more than an interview over the phone or a videoconferencing system, possibly with verbal queries and the use of home devices such as electronic thermometers. Somewhat more advanced approaches incorporate particular capabilities into individual add-on devices to existing computers or phones, but are piecemeal and unintegrated, and do not themselves provide any support for diagnostic work.

Embodiments of a medical data acquisition device described herein can be implemented using modern, cutting-edge advancements in sensor design and miniaturization, thereby enabling a single compact device to include multiple such sensors, making telemedical operations more practical. Embodiments of a medical data acquisition device described herein can be implemented using a modular design, thereby allowing additional components to be added when available and/or required. To this extent, the medical data acquisition device described herein can include provisions for additional sensors, and be configured to use both local and remote software to assist in evaluating the results from the sensors, thus supporting medical diagnostic work remotely.

A first aspect of the invention provides a medical data acquisition device comprising: a device housing; a plurality of medical sensors located on the device housing, wherein each medical sensor is configured to acquire a distinct type of diagnostic data for a patient; and a core component located in the device housing, wherein the core component includes: a plurality of sensor interfaces, wherein each sensor interface enables a medical sensor to be communicatively coupled to the core component; a data processing unit configured to receive raw diagnostic data from each of the plurality of medical sensors; and a communications component configured to provide medical diagnostic data for the patient for processing by an evaluation system.

A second aspect of the invention provides a patient assessment system comprising: a medical data acquisition device comprising: a device housing; a plurality of medical sensors located on the device housing, wherein each medical sensor is configured to acquire a distinct type of diagnostic data for a patient; and a core component located in the device housing, wherein the core component includes: a plurality of sensor interfaces, wherein each sensor interface enables a medical sensor to be communicatively coupled to the core component; a computer configured to receive raw diagnostic data from each of the plurality of medical sensors and generate medical diagnostic data for the patient based on the raw diagnostic data; and a communications component configured to provide the medical diagnostic data for the patient for processing by an assessment component; and a computing device including the assessment component, wherein the computing device is configured to communicate with the medical data acquisition device over a local communication link.

A third aspect of the invention provides a patient assessment system comprising: a medical data acquisition device comprising: a device housing; a plurality of medical sensors located on the device housing, wherein each medical sensor is configured to acquire a distinct type of diagnostic data for a patient, and wherein at least two of the plurality of medical sensors are configured to selectively use at least one of: a common sensing device or a common sensor accessory; and a core component located in the device housing, wherein the core component includes: a plurality of sensor interfaces, wherein each sensor interface enables a medical sensor to be communicatively coupled to the core component; a computer configured to receive raw diagnostic data from each of the plurality of medical sensors and generate medical diagnostic data for the patient based on the raw diagnostic data; and a communications component configured to provide the medical diagnostic data for the patient for processing by an assessment component; and a computing device including the assessment component, wherein the computing device is configured to communicate with the medical data acquisition device over a local communication link.

Other aspects of the invention provide methods, systems, program products, and methods of using and generating each, which include and/or implement some or all of the actions described herein. The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows an illustrative environment for evaluating a patient according to an embodiment.

FIG. 2 shows a more particular illustrative environment for evaluating a patient according to an embodiment.

FIG. 3 shows a more particular configuration of an illustrative medical data acquisition device according to an embodiment.

FIG. 4 shows an illustrative data flow diagram illustrating data processing features of the invention according to an embodiment.

FIG. 5 Shows an illustrative diagram of an example of prior typical configurations for remote medical data collection

FIG. 6 Shows an illustrative diagram of an expert system for medical evaluation

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As indicated above, aspects of the invention provide a medical data acquisition device including multiple medical sensors located on a single device housing. The medical sensors can be operated to acquire multiple types of diagnostic data for a patient. The device can include a core component that can interface with each medical sensor and receive raw diagnostic data from each medical sensor. The core component can generate medical diagnostic data for the patient based on the raw diagnostic data provide the medical diagnostic data for the patient for processing by an assessment component. The device can be utilized in a system that includes a local computing device capable of performing an assessment using the medical diagnostic data.

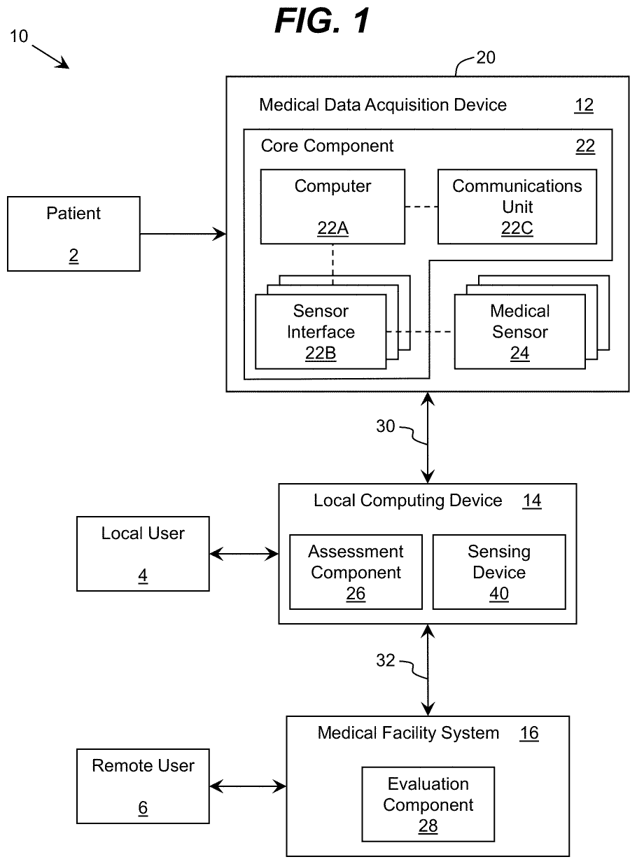

Turning to the drawings, FIG. 1 shows an illustrative environment 10 for evaluating a patient 2 according to an embodiment. To this extent, the environment 10 includes a medical data acquisition device 12 that can acquire diagnostic data for the patient 2 using any combination of a plurality of medical sensors 24 as part of a patient evaluation process described herein.

The medical data acquisition device 12 can include a device housing 20 which houses a core component 22 and a plurality of medical sensors 24. The core component 22 can include a computer 22A, a set of sensor interfaces 22B, and a communications unit 22C. The computer 22A can receive raw diagnostic data from a medical sensor 24 via a corresponding sensor interface 22B. The computer 22A can store the raw diagnostic data and/or process the raw diagnostic data (e.g., filter noise therefrom) and store the processed diagnostic data as medical diagnostic data acquired for the patient 2. The computer 22A can provide the medical diagnostic data for processing on a local computing device 14 via the communications unit 22C, which can have a local communications connection 30 with the local computing device 14.

The local computing device 14 can include an assessment component 26 that is configured to process the medical diagnostic data to generate an assessment of the patient 2. The assessment component 26 can provide the assessment to a local user 4, who can take one or more actions with respect to the patient 2 based on the assessment. Additionally, the assessment component 26 can transmit the medical diagnostic data and/or data corresponding to the assessment for processing on a medical facility system 16 using a remote communications link 32 between the local computing device 14 and the medical facility system 16.

The medical facility system 16 can include an evaluation component 28 which can present the medical diagnostic data and/or the assessment information for use by a remote user 6. Furthermore, the evaluation component 28 can process the data to further evaluate the patient 2. Additional evaluation information can be provided from the medical facility system 16 to the local computing device 14 for presentation to the local user 4, who can take one or more actions with respect to the patient 2 in response to the further evaluation information.

In general, the patient 2 is a human patient. However, it is understood that the medical data acquisition device 12 can be configured to obtain diagnostic data for any animal. In an embodiment, the local user 4 can be someone other than the patient 2, such as a caregiver (of a human or other animal), a first responder, a relative or friend of the patient 2, and/or the like. However, embodiments of the medical data acquisition device 12 can be configured for use by the patient 2. To this extent, references to patient 2 and local user 4 included herein include instances in which the patient 2 and local user 4 are the same individual. The remote user 6 can be any individual having more medical expertise than a typical person. To this extent, the remote user 6 can be any healthcare provider, including a nurse, a doctor, a first responder, a 911 operator, and/or the like, as well as individuals with healthcare training (e.g., volunteers) and individuals training to become a healthcare provider.

The medical data acquisition device 12 can comprise a single device housing 20 that integrates numerous medical sensors 24 in a design useful for home or field applications. The housing 20 can be sufficiently compact to enable its practical use in telemedical operations. To this extent, the housing 20 should be sufficiently small and the medical data acquisition device 12 should be sufficiently lightweight to enable a typical person, such as the local user 4, to transport the medical data acquisition device 12 between locations by, for example, carrying the medical data acquisition device 12.

The device housing 20 can be configured based on a target application for the medical data acquisition device 12. For example, when designed to be kept at a single location at which the medical data acquisition device 12 is anticipated to be utilized infrequently or used by a trained individual in a controlled environment, such as an office building, a clinic, a hospital, or the like, the device housing 20 can be a standard housing, which is capable of withstanding only minimal harsh use (e.g., drop from a short distance). However, when designed for field use, such as by a first responder, military use, home use by an untrained individual, or in other harsh environments, the device housing 20 and the corresponding components housed therein and thereon, can be ruggedized for better resistance to wear, stress, and abuse during use.

The core component 22 can be configured to enable the acquisition of diagnostic data of the patient 2 using the medical sensor(s) 24, and communication of medical data for processing by the local computing device 14. To this extent, the core component 22 can include a computer 22A, which can comprise any type of information processing system implemented using any type of hardware architecture capable of processing diagnostic data acquired by the medical sensors 24 and communicating with other systems using the communications unit 22C. For example, an embodiment of the computer 22A includes one or more processors as well as memory and/or data storage (e.g., a storage hierarchy). A processor can be a special purpose or general purpose processor. The computer 22A can be configured to execute program code which directs operation thereof and can be reprogrammable (e.g., via an update). However, an embodiment of the computer 22A can be configured to operate in a fixed manner, e.g., through hardware-implemented processing, a fixed program, and/or the like.

Each sensor interface 22B can comprise any type of interface for enabling communications and/or power supply between the computer 22A and a corresponding medical sensor 24. For example, a sensor interface 22B can be a standard or custom I/O interface, which can enable the serial or parallel communication of data between the computer 22A and the medical sensor 24. The sensor interface 22B also can enable the computer 22A to provide power for operating the medical sensor 24. An illustrative standard I/O interface is a universal serial bus (USB) port, which has become the generally dominant I/O interface; others include Apple's Thunderbolt, old-style parallel ports, and Ethernet connections. Additionally, a sensor interface 22B can enable wireless communication between the computer 22A and one or more medical sensors 24. For example, the sensor interface 22B can utilize a short-range wireless communication technology, such as Bluetooth, Wi-Fi, optical frequency, radio frequency, and/or the like, to enable communication between the computer 22A and the medical sensor(s) 24.

The communications unit 22C can be configured to enable a wired and/or wireless local communication connection 30 between the medical data acquisition device 12 and the local computing device 14. For example, the communications unit 22C can communicate with the local computing device 14 using a direct wired connection, such as using a USB connection. Additionally, the communications unit 22C can communicate with the local computing device 14 using a short-range wireless communication technology, such as Bluetooth, Wi-Fi, optical frequency, radio frequency, and/or the like.

A medical sensor 24 can comprise one or more of any type of sensing device capable of acquiring diagnostic data on a patient 2 that can be used in evaluating a condition of the patient. In an embodiment, the sensing device can comprise a standard sensing device. For example, illustrative sensing devices include a visible camera, a microphone, an accelerometer, and/or the like, each of which can be used to acquire certain diagnostic data for the patient 2. To this extent, in addition to medical sensors 24 located on the medical data acquisition device 12, the core component 22 can be configured to acquire diagnostic data from a sensing device located on another device. For example, the core component 22 can acquire diagnostic data from a sensing device 40 located on the local computing device 14. In an embodiment, the assessment component 26 can be configured to operate the sensing device 40 and/or direct the local user 4 to operate the sensing device 40 according to instructions received from the medical data acquisition device 12 in order to acquire the diagnostic data and provide the diagnostic data for processing by the core component 22.

A medical sensor 24 can include one or more emitting devices, which can be operated in conjunction with the sensing device(s). To this extent, a medical sensor 24 can include an imaging device, such as a visible or infrared camera. In this case, the medical sensor 24 may also include one or more visible or light emitting devices, which are configured for operation therewith. Similarly, a medical sensor 24 can include one or more microphones, and may also include one or more electroacoustic devices for generating acoustical energy. Additionally, a medical sensor 24 may be configured for operation in conjunction with one or more other medical sensors 24. For example, the medical sensors 24 can include multiple electrodes, such as wireless electrodes, which can be individually or concurrently used to acquire diagnostic data on the patient 2. It is understood that a medical sensor 24 can include various other types of sensing devices. Other illustrative sensing devices include a temperature sensor (e.g., an infrared sensor, a thermocouple, a thermistor, and/or the like), a chemical sensor, an accelerometer, a pressure sensor, an air flow sensor, etc.

Each medical sensor 24 can be configured to acquire diagnostic data targeted to acquire information regarding one or more of any of the various bodily systems including, for example, the cardiovascular system, the respiratory system, the immune system, the nervous system, the integumentary system, the visual system, the hearing system, etc. Illustrative medical sensors 24 include an electrocardiograph, an electroencephalograph, an oxygen saturation sensor (e.g., a pulse oximeter), a blood pressure sensor, a spirometer, an otoscope, an ophthalmoscope, a breath analyzer, a stethoscope, an ear thermometer, and/or the like. For some diagnostic data, a medical sensor 24 may include one or more accessories that facilitate placement of the sensing device and/or emitting device in a proper location for acquiring the diagnostic data. For example, an otoscope can include a speculum to enable examination of the ear. Similarly, an ophthalmoscope may be configurable for use with different lighting, lenses, aperture sizes, filters, and/or the like. Furthermore, various medical sensors, such as a spirometer or a breath analyzer, may include a mouthpiece for enabling the sensing of one or more attributes of the patient's 2 breathing.

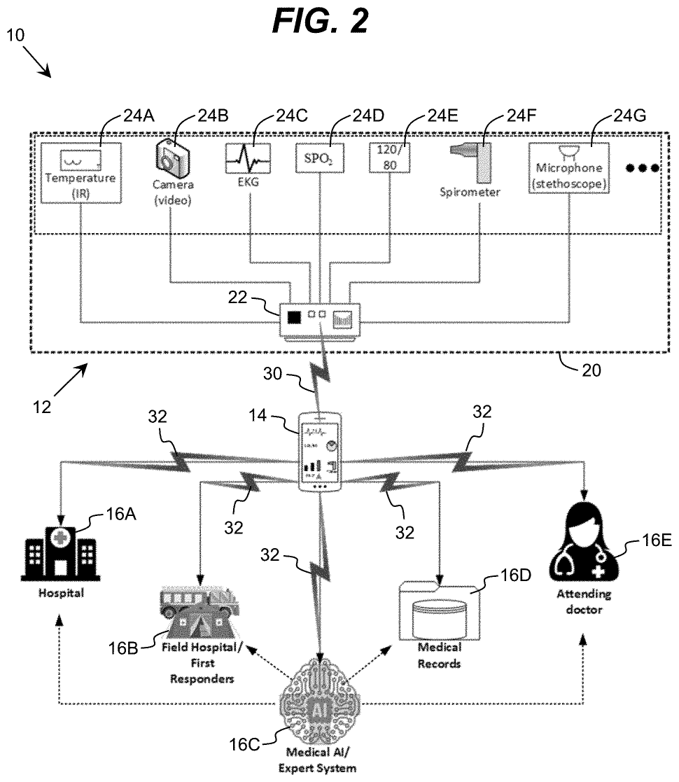

Aspects of embodiments of the invention are further described in conjunction with FIG. 2, which shows a more particular illustrative environment 10 for evaluating a patient 2 according to an embodiment. The medical data acquisition device 12 is shown including the core component 22 and a plurality of medical sensors 24A-24G all mounted to a device housing 20, which is conceptually illustrated by a dashed box. Additionally, the core component 22 is schematically shown as being operatively connected (e.g., via a wired or wireless connection) to each of the medical sensors 24A-24G mounted to the device housing 20.

In FIG. 2, the illustrative medical sensors 24A-24G include an infrared temperature sensor 24A, a video camera 24B, an electrocardiograph 24C, a blood oxygen saturation sensor 24D, a blood pressure sensor 24E, a spirometer 24F, and a microphone 24G. However, it is understood that additional medical sensors, alternative embodiments of the medical sensors 24A-24G, and/or alternative configurations of any combination of two or more medical sensors can be implemented. Each medical sensor 24A-24G can be mounted to the device housing 20 using any solution. For example, a medical sensor 24A-24G can be permanently mounted to a physical location of the device housing 20. Alternatively, a medical sensor 24A-24G and/or a component thereof (such as a disposable or removable accessory) can be stored in a particular location of the device housing 20 and removed for use in acquiring diagnostic data for the patient 2. In this case, the removable component may be reattached to the device housing 20 at a different location during use or may be placed on the patient 2 or at another location away from the device housing 20 during use.

The core component 22 can be configured to operate and acquire diagnostic data for the patient 2 using each of the medical sensors 24A-24G. Additionally, the core component 22 can process the raw diagnostic data to make the diagnostic data more usable in evaluating the patient 2. For example, the core component 22 can filter raw diagnostic data, enhance raw diagnostic data, and/or the like. Furthermore, the core component 22 can transform the raw diagnostic data from a native format into a format usable by an analysis system.

Additionally, the core component 22 can comprise a local communication link 30 (e.g., via a wired or wireless communications link) with a local computing device 14. In an embodiment, the local computing device 14 is a smartphone. However, it is understood that this is only illustrative of a mobile computing device, and any type of mobile computing device can be utilized, such as a laptop, a tablet computer, a special purpose computer, etc. Furthermore, for some applications of the environment 10, such as home monitoring of an individual, the local computing device 14 does not need to be a mobile computing device, but can be a desktop computer or the like.

Regardless, the local computing device 14 can provide more advanced data processing capabilities than the data processing capabilities of the core component 22. Additionally, the local computing device 14 can include a user interface and data presentation functionality for enabling a local user 4 (FIG. 1) to operate the medical data acquisition device 12, view medical data acquired by the medical data acquisition device 12, and/or the like. However, it is understood that the medical data acquisition device 12 also can include one or more user interfaces to enable use of the device 12 without requiring a connection to the local computing device 14.

Additionally, it is understood that the medical data acquisition device 12 can acquire diagnostic data from one or more sensing devices located on the local computing device 14. For example, the local computing device 14 can include a camera which can acquire image data provided to the medical data acquisition device 12 as diagnostic data. Similarly, the local computing device 14 can use accelerometers built into the local computing device 14 to acquire diagnostic data that can be used to evaluate movements of the patient 2.

In any event, the local computing device 14 can be equipped with a user interface and display for enabling the local user 4 to collect and view medical data of the patient 2. Additionally, the local computing device 14 can include an assessment component 26 (FIG. 1), which can be implemented as application software installed and executing on the local computing device 14. The assessment component 26 can be capable of providing a basic assessment of a condition of the patient 2 from the various vital statistics gathered using the medical data acquisition device 12.

In an embodiment, the assessment component 26 can perform an assessment based on medical diagnostic data acquired by a single medical sensor 24A-24G. For example, the assessment component 26 can determine if a measured value is higher or lower than an acceptable range of values. Similarly, the assessment component 26 can determine whether the medical diagnostic data includes any irregular features, such as irregular heartbeats, noisy or labored breathing, abnormal (e.g., missing or different) sounds, additional sounds, and/or the like. Illustrative assessments include a temperature of the patient as determined using the temperature sensor 24A being above or below a fever threshold, a blood pressure of the patient 2 as acquired by the blood pressure sensor 24E being over or under a key threshold for either diastolic or systolic pressure, and/or the like.

In an embodiment, the assessment component 26 also can perform an assessment using medical diagnostic data acquired by multiple medical sensors 24A-24G. For example, the assessment component 26 can be configured to evaluate a combination of: a high temperature as determined using the temperature sensor 24A; a low blood oxygen saturation as determined by the blood oxygen saturation sensor 24D; impeded breathing as determined by the spirometer 24F; and registered sounds of wheezing or rales as determiner by the microphone 24G (e.g., after use as a stethoscope), as indicating a preliminary diagnosis to be checked of respiratory infection of likely bacterial origin.

As illustrated, the local computing device 14 can be configured to communicate with one or more of various remotely located medical facility systems 16A-16E via remote communication links 32. It is understood that the medical facility systems 16A-16E shown are only illustrative of various medical facility systems 16A-16E with which the local computing device 14 can communicate. Regardless, each remote communications link 32 can be implemented using any combination of various communications protocols, wired and/or wireless transmission media, and/or the like. For example, the local computing device 14 can communicate with a medical facility system 16A-16E via a local computer network connected to the internet, transmissions over a cellular network, transmissions over a satellite network, a land mobile radio system, and/or the like.

For example, when the environment 10 is used for telemedicine, the local computing device 14 can communicate with a hospital 16A. The hospital 16A can be a hospital to which the patient 2 will be taken, if necessary, or the hospital 16A can be a distant hospital, such as one that has previously treated the patient 2, has some expertise in a known or suspected condition of the patient 2, and/or the like. Similarly, the local computing device 14 can communicate with a computer system of a field hospital or first responders 16B for evaluation by medical personnel located at the facility. This may occur in a military context, during a disaster, at the scene of an accident, or other situations, in which access to or connection with a regular hospital or doctor is not feasible. Additionally, the local computing device 14 can communicate with a computer system of a specific medical expert 16E, such as a particular attending doctor (e.g., as the specified provider for the patient 2 by the insurance policy), an attending doctor at a hospital, or otherwise a specific medical professional interested in and authorized for viewing the information provided.

In each case, a remote user 6, such as a nurse or doctor on duty, a first responder, the attending doctor, and/or the like, can use the corresponding computer system to receive and review the initial analysis from the local computing device 14 as well as the underlying medical diagnostic data. The remote user 6 can examine the vital signs provided and act on alerts, such as requesting the patient 2 have blood tests done, prescribing medicines for the patient 2 if they find they are confident of a diagnosis, and/or the like. Furthermore, the remote user 6 can conduct a video conference, telephone conversation, and/or the like, with the local user 4 and/or the patient 2 via the communications connection 30. For example, the remote user 6 can use such communications to perform an additional assessment of the patient 2, request the acquisition of additional medical diagnostic data, confirm proper operation of one or more medical sensors 24A-24G by the local user 6, and/or the like.

In an embodiment, the local computing device 14 can communicate with a medical diagnostic system 16C, such as a medical artificial intelligence (AI) or expert system. The medical diagnostic system 16C can provide additional computing resources for evaluating the patient 2, thereby providing further support for diagnosis and/or treatment of the patient 2. In a more particular embodiment, the medical diagnostic system 16C can be configured to operate in cooperation with multiple instances of the local computing device 14 and medical data acquisition device 12 to provide additional diagnostic capabilities. It should be obvious to anyone familiar with the art that other types of medical artificial intelligence algorithms and approaches including but not limited to convolutional neural networks, deep learning, etc. can be used without deviating from this invention.

The medical diagnostic system 16C can further communicate with one or more of the other remote medical systems 16A-16B, 16D-16E, in order to diagnose other patients, improve the algorithms with which the medical diagnostic system 16C performs the evaluation, disseminate information derived from evaluations, and/or the like. In an embodiment, the medical diagnostic system 16C can include an evaluation component 28 (FIG. 1) which uses the data acquired from multiple medical data acquisition devices 12 and/or other medical data sources to search for public health patterns, trends in individual health, analyze the data with super computers to deduce complex decisions with the help of AI, and/or the like. The results of such analysis can be pushed out to the assessment component 26 to improve the local assessment of the patient 2. For example, the analysis by the evaluation component 28 can detect the spread of a contagion in a geographic area. In response, the evaluation component 28 can provide instances of the assessment component 26 in and near the geographic area information to enable the assessment components 26 to assess patients 2 exhibiting symptoms of the contagion.

The assessment component 26 in the local computing device 14 may incorporate its own medical expert system AI, in addition to the external expert system 16c. An expert system is, as the name implied, a system that incorporates the knowledge of one or more experts in the field that has been carefully reduced to sets of rules which may include simple yes-no evaluations but more often include weighted probability branchings which may combine in complex ways to produce a diagnosis of a condition. These rules and conditionals are able to be updated as needed.

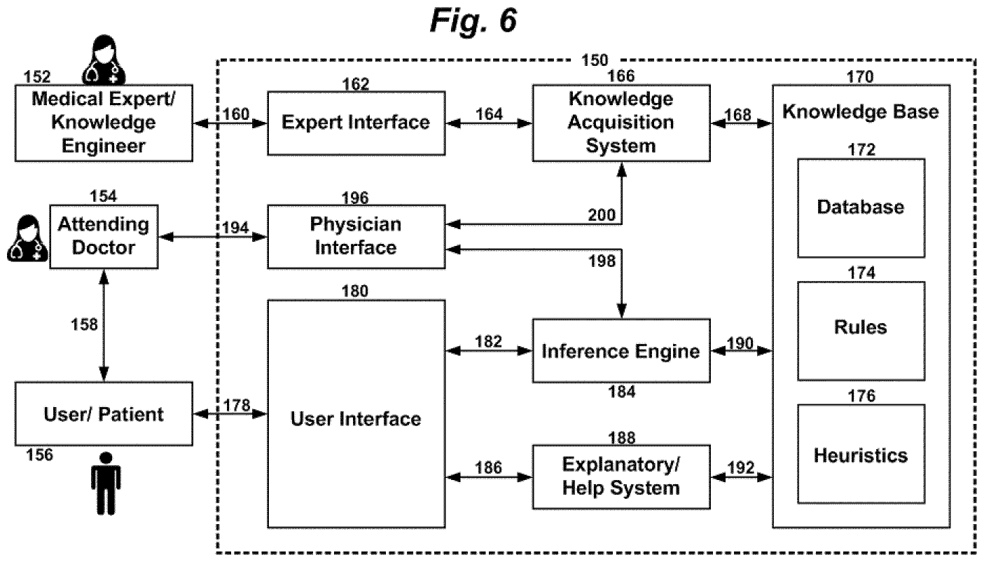

The structure and use of a medical expert system is shown in more detail in FIG. 6. The expert system 150 may be informed by or interact with medical experts or knowledge engineers 152, attending doctors 154, and end users/patients 156. Attending doctors 154 and end users 156 may also communicate 158; said communications 158 may include face-to-face office visits, teleconferences, phone calls, emails, data transfer from the proposed invention to the doctor's office, or any other means of communication.

In any event, the creation and maintenance of the expert system 150 is performed primarily (though not exclusively) by knowledge engineers/medical experts 152, who can interact 160 with an expert interface 162, which exchanges information 164 with a knowledge acquisition system 166; the knowledge acquisition system 166 communicates 168 with the core of the expert system 150, the knowledge base 170. The knowledge base 170 is generally comprised of an overall database 172, sets of rules 174, and heuristics 176. The knowledge acquisition system 166 may create, modify, or remove material from any portion of the knowledge base 170; this may be performed based on specific instructions from the medical expert/knowledge engineer 152, or on a self-learning capability included in the knowledge acquisition system 166. An example of the latter would be a Bayesian network which could take inputs such as the outcome of specific advice from the expert system 150 and use it to modify probabilities in the rules 174 to improve later outcomes.

For the purposes of the present invention, and assuming an expert system installed on, or in communication with, the computing device 14, the user/patient 156 will be the primary actor interacting with the expert system 150 following its creation. This is done through communication 178 with the user interface 180; such communication 178 may involve direct input of health data from available sensors 24, selections from a standard touchscreen menu, voice-activated commands, typed data input, or any other means by which the user 156 could provide information or instructions to the user interface 180.

The user interface 180 also has a connection 182 to an inference engine 184 and another connection 186 to an explanatory or help system 188. The inference engine 184 performs the actual work of evaluation and diagnosis of conditions, making use 190 of the knowledge base 170; the engine 184 may access the database 172, apply rules 174 based on the user input 178, or apply heuristics 176 as needed.

The explanatory/help system 188 may also communicate with the knowledge base to provide answers to operational queries by the user 156; these may include queries of procedure, medical terms, operation of the system, and so on.

The attending doctor 154 may also make use of the expert system 150 through operating 194 a physician interface 196. This interface allows the physician to request and receive 198 inferences from the inference engine 184; it may also allow the physician 152 to provide input 200 to the knowledge acquisition system 166, especially if the knowledge acquisition system 166 is provided with a self-learning and updating capability; the physician 152 might, for instance, provide the knowledge acquisition system 166 with updates on the accuracy of the expert system 150's performance, on the association of specific systems with particular conditions, and so on.

The output of the expert system 150 will vary depending on the user, on the certifications and capabilities of the system, and so on. These may thus range from simple notifications to the user 156 to contact their physician or to go to the local emergency room, to detailed evaluations of health, existing or likely illnesses, and recommended courses of treatment to an attending physician 154.

In any event, the local computing device 14 also can communicate with a computer system managing a database of medical records 16D. For example, the database of medical records 16D may be a personal database of records for the individual patient 2. Such a personal database can be maintained at the doctor's office of the patient 2 or by a third party, which can maintain a database (e.g., local, regional, or national) for numerous instances of the medical data acquisition device 12. In this case, the database of medical records 16D can be used for any of various purposes, such as to maintain a health history of a patient 2, which can be selectively disseminated to medical personnel when treating the patient 2. Additionally, the database of medical records 16D can maintain the medical diagnostic data (e.g., after removing any potentially personally identifiable information) for any of various purposes, as desired by the owner and operator of the environment 10, such as improving diagnoses, training users and/or algorithms, and/or the like.

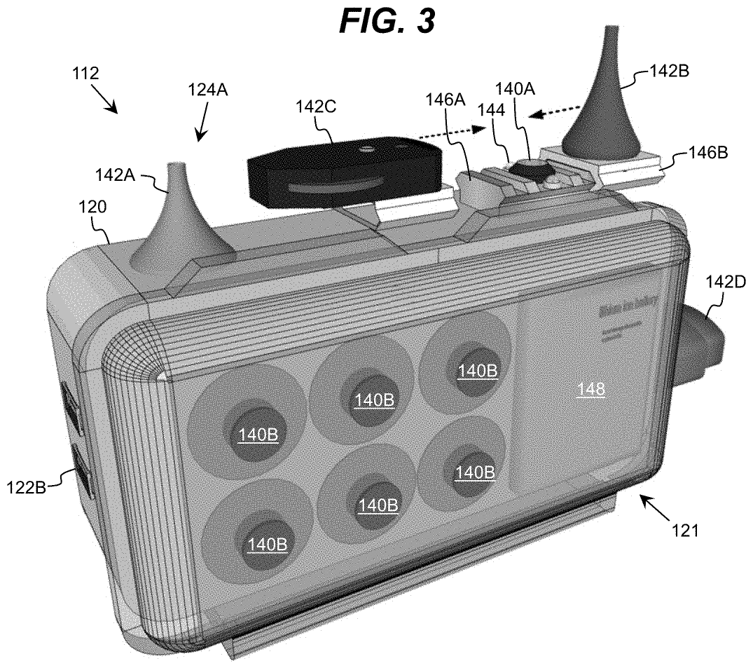

FIG. 3 shows a more particular configuration of an illustrative medical data acquisition device 112 according to an embodiment. It is understood that the medical data acquisition device 112 is only illustrative, and embodiments of the medical data acquisition device 112 described herein can be configured with a different physical design, different default sensors, different sensor connectors, different methods of use, etc.

Regardless, the medical data acquisition device 112 includes a main device housing 120 which is equipped with various medical sensors and other components required for operation of the medical data acquisition device 112. For example, the medical data acquisition device 112 is shown including a set of charging/data ports 122B, each of which can enable connection of medical sensor thereto, communication with the local computing device 14 (FIG. 1), power to be provided for operating the medical data acquisition device 112 and/or recharging a set of batteries included therein, and/or the like.

In this embodiment, the medical data acquisition device 112 includes an ear thermometer 124A, which can include a speculum 142A and a corresponding temperature sensing device (e.g., an infrared temperature sensor). The speculum 142A can be permanently or removably affixed to a location on an outer surface of the device housing 120 using any solution. In an embodiment, the device housing 120 can include storage space for one or more accessories to clean the speculum 142A between uses, cover the speculum 142A when not in use, and/or the like. Furthermore, when removable, the speculum 142A can be designed for a single use and subsequent disposal and the device housing 120 can include storage space for multiple specula 142A.

An embodiment of the medical data acquisition device 112 can enable the reuse of one or more sensing devices and/or sensor accessories in multiple types of medical sensors. For example, a camera 140A is shown mounted to the device housing 120 with a lens located on the exterior surface of the device housing 120. Additionally, the device housing 120 can include one or more light emitting devices 144 mounted adjacent to the camera 140A. The light emitting devices 144 can be configured to be selectively used in conjunction with the camera 140A to provide illumination to an area being imaged by the camera 140A. In an embodiment, a light emitting device 144 can be a light emitting diode. In a more particular embodiment, the light emitting device 144 is configured to emit visible light, such as white light. However, it is understood that this is only illustrative of possible embodiments of the light emitting device 144, which can be configured to emit light perceived as a certain color, infrared light (e.g., when the camera 140A is sensitive to infrared radiation), and/or the like. Regardless, the light emitted by the light emitting device 144 can be diffuse or collimated.

In an embodiment, the device housing 120 can enable the camera 140A (or another sensing device) to be incorporated into multiple different medical sensors. To this extent, the device housing 120 is shown including a removable mounting system 146 which enables a sensor accessory to be selectively mounted over the camera 140A and light emitting device(s) 144 and selectively removed therefrom. For example, the removable mounting system 146 can include a rail 146A affixed to the surface of the device housing 120 next to the camera 140A. The rail 146A can be configured to accept a complementary mount 146B, which can be slid over the rail 146A and temporarily secured into the appropriate location with respect to the camera 140A and/or light emitting device(s) 144 using any solution, such as a stop mechanism located on the mount 146B or the rail 146A.

However, it is understood that a rail mounting system is only illustrative of various mounting systems that can be utilized to temporarily mount an accessory with proper alignment with a sensing device. For example, other illustrative mounting systems include a screw-threaded mount, a bayonet mount, a breech-lock (friction lock) mount, and/or the like. Additionally, a mount can use magnetic force to assist with aligning and/or maintaining alignment. The medical data acquisition device 112 can include a mechanism for storing sensor accessories when they are not in use. For example, the medical data acquisition device 112 can include a storage compartment with one or more sets of rails 146A, which can enable the sensor accessories to be secured for storage when not in use.

As illustrated, multiple sensor accessories, such as sensor accessories 142B, 142C, can be configured for temporary mounting and use with the camera 140A. For example, the medical data acquisition device 112 can include a speculum 142B mounted to the mount 146B, which can be mounted and aligned with the camera 140A. In this configuration, the speculum 142B, camera 140A, and light emitting device(s) 144 can be operated as an otoscope to acquire image data of the ear, nose, or throat of the patient 2. In an embodiment, the speculum 142B can be removably mounted to the mount 1466 and replaced, cleaned between uses, covered when not in use, and/or the like, as described herein.

Similarly, the medical data acquisition device 112 can include an ophthalmoscope accessory 142C, which includes a mount for temporarily mounting the ophthalmoscope accessory 142C above and in alignment with the camera 140A and light emitting device(s) 144. The ophthalmoscope accessory 142C can be used to enable imaging of the eye of the patient 2 using any combination of different configurations of magnification (e.g., by the selection of different lenses), lighting (e.g., varying brightnesses, as well as white or colored light by the selection of desired filters), and/or the like.

In this manner, any medical sensor requiring a camera 140A for use in acquiring diagnostic data for the patient 2 can be incorporated into the medical data acquisition device 112. Regardless, it is understood that the camera 140A also can be operated without an accessory to acquire various types of diagnostic data for the patient 2. For example, the local user 4 can operate the camera 140A (with or without the light emitting device 144) to acquire image data of a region of the skin of the patient 2, which may have a rash or other indication of concern.

Additionally, the medical data acquisition device 112 can enable a sensor accessory to be utilized by multiple medical sensors. For example, the device housing 120 is shown including a mouthpiece accessory 142D mounted on a surface thereof. In an embodiment, the mouthpiece accessory 142D can be removably mounted using a solution described herein. When mounted, the mouthpiece accessory 142D can be properly aligned and configured for use with one or more sensing devices which acquire spirometric data (e.g., airflow speed, pressure, and/or the like) when the mouthpiece accessory 142D is used as part of a spirometer. Additionally, the mouthpiece accessory 142D can be properly aligned and configured for use with one or more chemical sensing devices (e.g., carbon dioxide, alcohol, etc.) when the mouthpiece accessory 142D is used as part of a breath analyzer. In such a configuration, it is understood that the mouthpiece accessory 142D can be concurrently used as part of both a spirometer and a breath analyzer, thereby enabling the concurrent acquisition of multiple types of medical diagnostic data for the patient 2.

As discussed herein, the device housing 120 can include one or more compartments for storage of various sensors, sensing devices, sensor accessories, device accessories, and/or the like. To this extent, the device housing 120 is shown including a side compartment 121, which can be configured to store various components. For example, the side compartment 121 is shown including a plurality of electrodes 140B. In an embodiment, the electrodes 140B can comprise wireless electrodes. Regardless, the electrodes 140B can be used to enable the medical data acquisition device 112 to obtain an EKG, EEG, or acquire diagnostic data for other behaviors of neuromuscular systems of the patient 2. While not shown, it is understood that the side compartment 121 or another compartment located on the device housing 120, also can store other medical sensors, such as a solid state (cuffless) blood pressure monitor, a blood sugar sensor, and/or the like. Furthermore, a side compartment 121 can include other accessories, such as maintenance/support materials, a spare battery 148, etc.

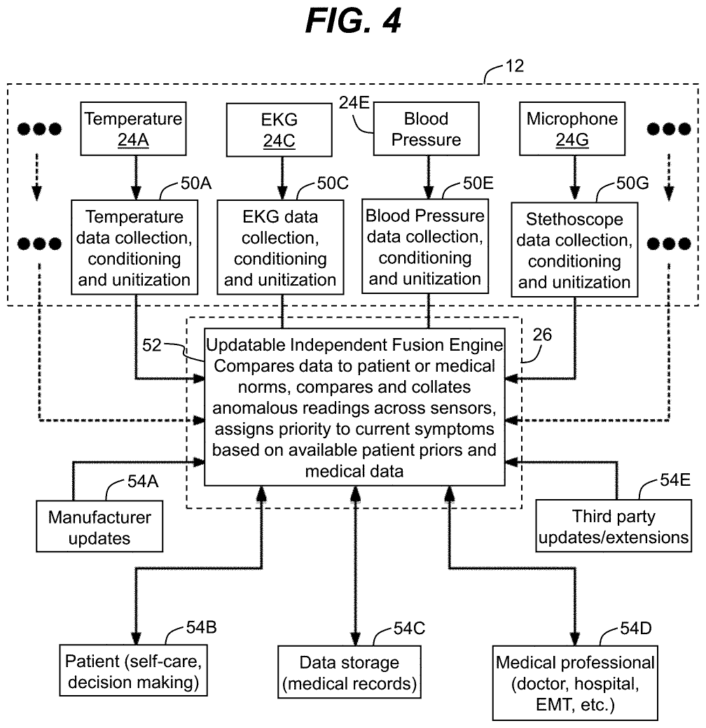

FIG. 4 shows an illustrative data flow diagram illustrating data processing features of the invention according to an embodiment. As illustrated, each medical sensor 24A, 24C, 24E, 24G of the medical data acquisition device 12 can provide raw diagnostic data for processing by a corresponding diagnostic data processing component 50A, 50C, 50E, 50G, each of which is at least partially implemented on the core component 22 (FIG. 1) of the medical data acquisition device 12.

In an embodiment, each data processing component 50A, 50C, 50E, 50G is configured to process raw diagnostic data specific to an individual medical sensor 24A, 24C, 24E, 24G. Such a configuration can be useful when the raw diagnostic data acquired by each medical sensor 24A, 24C, 24E, 24G has unique characteristics in the manner in which it is acquired and/or processed from the raw diagnostic data acquired by the other medical sensors 24A, 24C, 24E, 24G. However, it is understood that this is only illustrative, and a data processing component 50A, 50C, 50E, 50G can be configured to process the raw diagnostic data acquired by multiple medical sensors 24A, 24C, 24E, 24G. For example, when two or more medical sensors acquire image data, the same data processing component can be used to process the raw diagnostic data.

More particularly, the data processing component 50A can be configured to collect and process temperature data using the medical sensor 24A by performing many very quick measurements and averaging the measurements to produce a more accurate reading. Subsequently, the data processing component 50A can convert the raw temperature data into a standard temperature format, such as degrees Fahrenheit or Celsius.

To generate EKG data using the medical sensor 24C, the data processing component 50C can acquire multiple raw electrical readings from different locations on the body of the patient 2. Each such measurement will have electronic noise, which the data processing component 50C can filter. Subsequently, the data processing component 50C can combine the filtered readings according to standard EKG rules across the time domains to generate a full EKG signature.

The data processing component 50E can acquire raw blood pressure data (e.g., data corresponding to blood flow) using the medical sensor 24E. The data processing component 50E can determine readings for both diastolic and systolic pressure from the raw blood pressure data and convert the readings to standard blood pressure measurements, such as in millimeters of mercury (mm Hg).

For acquiring data using the microphone 24G (e.g., when used as a stethoscope), the data processing component 50G can direct the local user 4 regarding placement of the microphone 24G on the body of the patient 2. Subsequently, the data processing component 50G can acquire raw audio data and perform filtering and/or enhancements of the acoustic signals to generate medical diagnostic data suitable for analysis. The analysis can include, for the lungs, detecting standard breathing sounds, wheezing, rales, and/or the like. Similar analysis can be performed for other locations on the body, such as the digestive tract. In embodiments, the analysis can be manually performed or performed by the assessment component 26 and/or a medical facility system 16.

While each data processing component 50A, 50C, 50E, 50G is illustrated as being implemented on the medical data acquisition device 12, it is understood that the functionality described in conjunction with a data processing component 50A, 50C, 50E, 50G can be implemented using a combination of the core component 22 of the medical data acquisition device 12 and the assessment component 26 of the local computing device 14.

In any event, the medical diagnostic data generated by each data processing component 50A, 50C, 50E, 50G can be further processed by the assessment component 26 of the local computing device 14. In an embodiment, the assessment component 26 includes an updatable independent fusion engine 52 to perform an assessment of the patient 2. The fusion engine 52 can incorporate multiple solutions to extract an understanding from the medical diagnostic data received from the data processing components. For example, the fusion engine 52 can compare measurement data to standard norms, established norms for the patient 2, and/or the like. Furthermore, the fusion engine 52 can perform a more detailed analysis of the data, e.g., by combining the data and results from more than one parameter to arrive at more comprehensive conclusions and diagnoses.

For example, initially the temperature sensor 24A, after initial collection and processing by the data processing component 50A, might return a temperature of 98.9 degrees Fahrenheit. Using a standard threshold of, for example, 99.5 degrees, the fusion engine 52 may not identify any anomaly. However, patient history over months may indicate that the patient's temperature has ranged between 96.5 and 97.8, never passing 98 degrees. In this case, the fusion engine 52 can identify a difference of more than a degree from the highest prior measurement of the healthy patient 2, and can trigger an evaluation of a mild fever for the patient 2.

History can be applied in a more nuance fashion as well. For example, it is well-known that temperature of individuals generally fluctuates throughout the day, with the lowest temperatures usually seen during and immediately after sleep and highest at late afternoon or the equivalent). The fusion engine 52 could evaluate the patient 2's temperature history and see that the temperature of 98.9 is being taken immediately after patient 2 awakened, and thus increase the certainty of patient 2 having a fever, as the expected temperature upon awakening was 96.8.

In a fusion context, when the fusion engine 52 evaluates the patient 2 as having a fever from the temperature data, and also evaluates the patient 2 as having an inflamed throat (e.g., from image data acquired using an otoscope) and detects wheezing from data acquired by the stethoscope 24G, the fusion engine 52 can flag a possible diagnosis of a respiratory infection, at least upper and possibly descending to middle or lower respiratory tract. In an embodiment, the fusion engine 52 can incorporate a small to moderate-sized medical expert system to assist in diagnostic operations in the field or at home. This expert system could provide advisories to the patient 2 as to the need to seek expert medical attention, and then could provide evaluations as well as sensor data to the physician.

As indicated, the assessment component 26, and more particularly the fusion engine 52, can be configured to be flexible and updatable. In this manner, the assessment component 26 can take advantage of improvements and advances in automatic diagnostic and medical analysis data. For example, the assessment component 26 and/or the fusion engine 52 can be updated by the manufacturer 54A, which can also incorporate specifications for new sensor data collection modes as well as additions to the fusion engine 52 itself. In the former case, when appropriate, the assessment component 26 can forward revised or updated specifications received from the manufacturer 54A to the medical data acquisition device 12 for implementation thereon (e.g., by the core component 22). Similarly, updates and/or extensions from a properly vetted third party 54E can be provided to update the assessment component 26, the fusion engine 52, and/or the medical data acquisition device 12. Such updates or extensions can be used to address a special application for which the medical data acquisition device 12 is being utilized. For example, the medical data acquisition device 12 may be being used for home monitoring of a patient 2 with long term health issues with specific conditions for which monitoring is desired.

In addition, the assessment component 26 and/or the fusion engine 52 may interact with external individuals/systems to refine, extend, and/or improve its performance through feedback from others, and through the ability to reference historic data as well. As illustrated, the interaction can include direct interaction with a patient 54B, e.g., as part of their own self-care and decision making, such as whether to visit a local urgent care facility, contact a doctor, go to an emergency room, stay home, etc. Similarly, the assessment component 26 and/or the fusion engine 52 can directly interact with a medical professional 54D (e.g., an evaluation component 28 (FIG. 1) of the medical professional 54D), such as the primary care doctor of the patient, staff of a hospital emergency room, emergency medical technician, and/or the like. Such professionals can provide feedback regarding how well the fusion engine 52 evaluations matched a final diagnosis, details of how the fusion engine 52 diagnosis was refined to obtain the final diagnosis, etc. The fusion engine 52 can use the feedback to refine its evaluation procedures through any suitable feedback mechanism.

Furthermore, the assessment component 26 and/or the fusion engine 52 can transmit and/or receive data and evaluations to or from data storage 54C which includes the medical records of the patient. The data storage 54C can be stored locally, e.g., on the local computing device 14, for local personal medical data storage. Alternatively, the data storage 54C can be located on the medical facility system 16, e.g., at the primary care physician's office, a central repository for medical data, and/or the like. Access to the medical data storage 54C can be strictly controlled using known solutions.

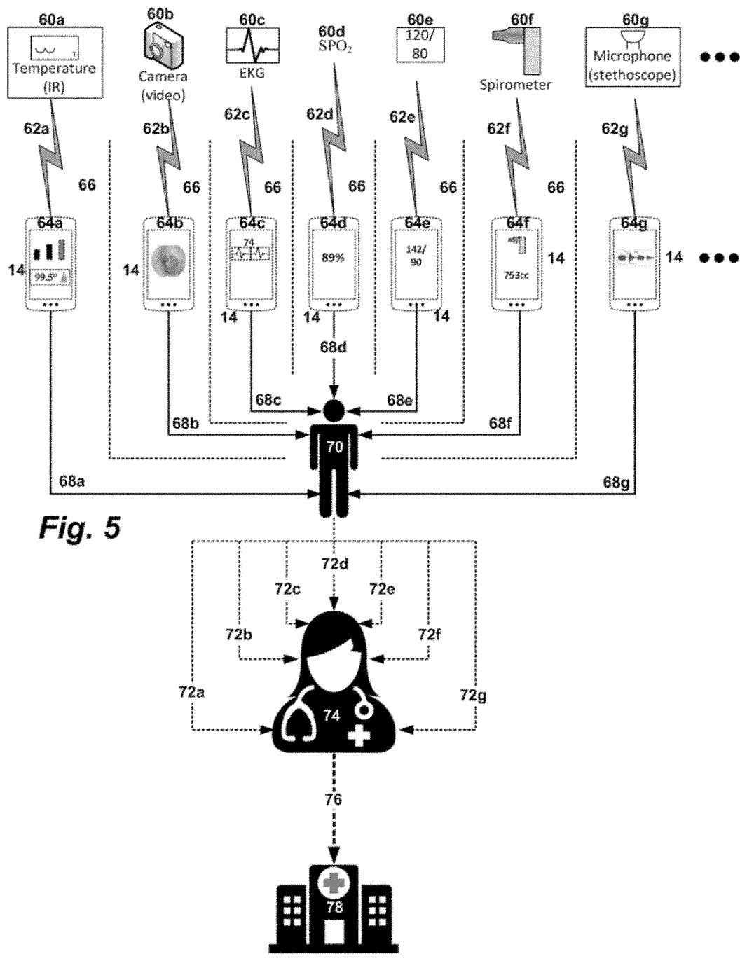

The described invention addresses key shortcomings of existing technology, some of which are worth describing in detail now that the invention itself is presented. FIG. 5 illustrates limitations of current technology approaches to any attempt to imitate the functionality of the present invention. Multiple separate sensing devices for medical parameters 60a through 60g are configured to connect 62a through 62g with their corresponding applications 64a through 64g on a local computing device 14. Because these devices 60a-g are separately purchased, possibly from multiple vendors, there exist barriers 66 to their cooperative and efficient use. These barriers may be physical (each has its own recharging or power source, each may use different communication technologies) or programming/procedural (each uses a separate custom application 64a-g, data is transmitted using different and noncompatible protocols, each must be separately connected with the device 14 by, e.g., Bluetooth pairing, the individual applications 64a-g cannot communicate with each other, etc.).

This differs drastically from the present invention, in which the devices 24a-g use the same connection and protocol methods and software, and use integrated, common power/recharging and physical interfaces.

In any event, because of barriers 66, medical parameter measurements 68a through 68g are individually passed to the user 70 of these separate devices 60a-g. The individual applications 64a-g may or may not perform some evaluation of the data (for example, the SPO₂ meter 60d may sound an alarm when oxygen levels fall below some threshold), but cannot exchange or compare information between applications. Most separate devices 60a-g also provide minimum history recording or analysis.

This is very different from the present invention, as shown by FIGS. 2 and 4, in which it is clear that data is received and presented simultaneously or cumulatively in the provided application, and in which evaluation of the data of all sensors may be performed to specified and varying degrees by the Fusion Engine 52. This fusion may involve simple linking of measurements/symptoms (for example, a fever combined with low SPO₂ and abnormal breathing sounds) or a combination of symptoms and history.

Continuing with FIG. 5, user 70 thereby generally becomes the recipient and repository of the data from the separate sensors 60a through 60g. Through a means (telephone, teleconference, email, etc.) the user 70 may provide this data 72a through 72g to their physician 74, but almost always through verbal or written means; few of the separate commercial devices 60a-g provide a method within their applications 64a-g to transfer data to a separate remote system such as a doctor's office. This introduces an increased likelihood of human error in the user 70's actions in providing their physician 74 with medical data, especially in the case of medical data unfamiliar to the layman (spirometer, EKG, stethoscope readings, etc.).

Similarly, while the physician 74 is far more capable of proper understanding and diagnosis than their patient 70, they too are capable of human error when entering and transmitting their evaluation and data 76 to the hospital records 78.

All of these failings are potential barriers to the proper, quick, and efficient treatment of any health issues faced by patient 70, and all of these limitations are addressed by the current invention, as described previously.

Returning to FIG. 1, as described herein, the medical data acquisition device 12 can provide a mobile device with various medical sensors located thereon. However, it is understood that the medical data acquisition device 12 can be configured to interact with one or more medical sensors implemented apart from the medical data acquisition device 12. For example, the medical data acquisition device 12 can be configured to interface with an X-ray machine, ultrasound unit, CT scanner, and/or the like, when such an instrument is locally available and can be communicated with using a local communication connection 30 as described herein. Similarly, the assessment component 26 can be configured to acquire medical data acquired by such other systems from a medical facility system 16, such as the medical records of the patient 2, and use such medical data in conjunction with the medical diagnostic data acquired by the medical data acquisition device 12 in evaluating the patient 2.

In an illustrative application, the medical data acquisition device 12 can be utilized by a patient 2 to self-monitor his/her health over a period of time. For example, the medical data acquisition device 12 described herein can be configured to enable the patient 2 to easily monitor and track key health indicators (e.g., heart rate before, during, and after exercise, oxygen levels, blood pressure, overall activity, etc.) and log them to the local computing device 14 or a medical facility system 16 equipped with some version of a medical assessment component 26 as described herein. The medical assessment component 26 can track the performance of the patient 2 over time and be able to detect changes in endurance, activity, and vital medical statistics in a way that permits early detection and screening for more serious conditions, long before the patient 2 would be likely to notice.

In another illustrative application, the medical data acquisition device 12 can be utilized in emergencies and in settings such as battlefields, or during major disasters (tsunami, hurricane, earthquakes, pandemics) where it is frequently impractical to bring a patient 2 to a standard hospital or primary care doctor. In particular, there may be no functional medical facilities on hand, or such facilities may be currently overloaded with patients and unable to take on more load. In this case, the medical data acquisition device 12 and assessment component 26 described herein can permit a superior level of triage and treatment recommendations, especially with a suite of medical sensors 24 to support the specific issues expected in the disaster or emergency setting. The medical data acquisition device 12 and assessment component 26, then, would be a primary evaluation and diagnostic tool for professionals or amateurs pressed into emergency service.

As used herein, unless otherwise noted, the term “set” means one or more (i.e., at least one) and the phrase “any solution” means any now known or later developed solution. The singular forms “a,” “an,” and “the” include the plural forms as well, unless the context clearly indicates otherwise. Additionally, the terms “comprises,” “includes,” “has,” and related forms of each, when used in this specification, specify the presence of stated features, but do not preclude the presence or addition of one or more other features and/or groups thereof.

The foregoing description of various embodiments of this invention has been presented for purposes of illustration and description. It is not intended to be exhaustive or to limit the invention to the precise form disclosed and inherently many more modifications and variations are possible. All such modifications and variations that may be apparent to persons skilled in the art that are exposed to the concepts described herein or in the actual work product, are intended to be included within the scope of this invention disclosure.

What is claimed is:

1. A medical data acquisition device comprising:
a device housing;
a plurality of medical sensors, wherein each medical sensor is configured to acquire diagnostic data for a patient; and
a core component located in the device housing, wherein the core component includes:
a plurality of sensor interfaces, wherein each sensor interface enables a medical sensor to be communicatively coupled to the core component;
a computer configured to receive raw diagnostic data from each of the plurality of medical sensors and generate medical diagnostic data for the patient based on the raw diagnostic data; and
a communications component configured to provide the medical diagnostic data for the patient for processing by an assessment component.

2. The device of claim 1, wherein the plurality of medical sensors include at least one sensor selected from: an electroencephalograph, an oxygen saturation sensor, a blood pressure sensor, a spirometer, an otoscope, an ophthalmoscope, a breath analyzer, a stethoscope, and body temperature measurement device.

3. The device of claim 1, wherein the device can communicate with at least one of the following, either installed on the device or on a remote system: a medical diagnostic system, a medical artificial intelligence system, and an expert system.

4. The device of claim 1, wherein the computer is configured to filter noise from the raw diagnostic data of at least one of the plurality of medical sensors and store the filtered raw diagnostic data as the medical diagnostic data for the patient.

5. The device of claim 1, wherein at least one of the plurality of medical sensors is mounted in or to an external surface of the device housing.

6. The device of claim 5, wherein at least one sensor mounted in the housing comprises at least one removably mounted sensor.

7. The device of claim 6, wherein the device further comprises a mounting object for at least one removably mounted sensor.

8. The device of claim 1, wherein at least one of the plurality of medical sensors includes a camera.

9. The device of claim 8, wherein the at least one of the plurality of medical sensors further includes a set of light sources mounted adjacent to the camera and configured for operation in conjunction with the camera.

10. The device of claim 1, wherein at least of the sensor interfaces comprises at least one interface adapted to receive wired signals or wireless signals.

11. The device of claim 1, wherein at least one the plurality of medical sensors includes a mouthpiece accessory mounted to an external surface of the device housing.

12. The device of claim 11, wherein the at least one the plurality of medical sensors comprises a spirometer.

13. The device of claim 11, wherein the at least one the plurality of medical sensors includes a plurality of chemical sensors configured for use in conjunction with the mouthpiece accessory.

14. A patient assessment system comprising:
a medical data acquisition device comprising:
a device housing;
a plurality of medical sensors, wherein each medical sensor is configured to acquire a distinct type of diagnostic data for a patient; and
a core component located in the device housing, wherein the core component includes:
a plurality of sensor interfaces, wherein each sensor interface enables a medical sensor to be communicatively coupled to the core component;
a computer configured to receive raw diagnostic data from each of the plurality of medical sensors and generate medical diagnostic data for the patient based on the raw diagnostic data; and
a communications component configured to provide the medical diagnostic data for the patient for processing by an assessment component; and
a computing device including the assessment component, wherein the computing device is configured to communicate with the medical data acquisition device.

15. The system of claim 14, wherein the assessment component manages an interface to enable a local user to use the medical data acquisition device to acquire the medical diagnostic data for the patient.

16. The system of claim 14, wherein the assessment component includes a fusion engine to assess a condition of the patient using the medical diagnostic data acquired by at least two of the plurality of medical sensors.

17. The system of claim 16, wherein the assessment component is configured to adjust operation of the fusion engine in response to an evaluation of an accuracy with which the fusion engine assessed the condition

18. The system of claim 14, wherein the system can communicate with at least one of the following, which may be installed on the medical data acquisition device or on a remote device: a medical diagnostic system, a medical artificial intelligence system, and an expert system.

19. A patient assessment system comprising:
a medical data acquisition device comprising:
a device housing;
a plurality of medical sensors, wherein each medical sensor is configured to acquire a distinct type of diagnostic data for a patient, and wherein at least two of the plurality of medical sensors are configured to selectively use at least one of: a common sensing device or a common sensor accessory; and
a core component located in the device housing, wherein the core component includes:
a plurality of sensor interfaces, wherein each sensor interface enables a medical sensor to be communicatively coupled to the core component;
a computer configured to receive raw diagnostic data from each of the plurality of medical sensors and generate medical diagnostic data for the patient based on the raw diagnostic data; and
a communications component configured to provide the medical diagnostic data for the patient for processing by an assessment component; and
a computing device including the assessment component, wherein the computing device is configured to communicate with the medical data acquisition device.

20. The system of claim 19, wherein the system can communicate with at least one of the following, which may be on the medical data acquisition device or on a remote device: a medical diagnostic system, a medical artificial intelligence system, and an expert system.
